(19) United States
(12) Patent Application Publication
(10) Pub. No.: US 2003/0118993 A1
(43) Pub. Date: Jun. 26, 2003

(54) Nucleic acid which is upregulated in human tumor cells, a protein encoded thereby and a process for tumor diagnosis

(76) Inventors: Sepp Kaul, Wiesloch (DE); Josef Preiherr, Weilheim (DE); Erika Karolina Preiherr, Weilheim (DE); Josef Preiherr, Habach (DE); Ingrid Preiherr, Habach (DE); Ulrich Weidle, Muenchen (DE)

Correspondence Address: HOFFMANN-LA ROCHE INC., PATENT LAW DEPARTMENT, 340 KINGSLAND STREET, NUTLEY, NJ, 07110

(21) Appl. No.: 09/864,954

(22) Filed: May 24, 2001

(30) Foreign Application Priority Data
May 26, 2000 (EP) 00110953.7
Jul. 15, 2000 (EP) 00115369.1

(51) Int. Cl. C12Q 1/68

(52) U.S. Cl. 435/6

(57) ABSTRACT

A nucleic acid molecule (PKW) with the nucleic acid sequence SEQ ID NO:1 is upregulated in mammary carcinoma cells. The PKW protein of SEQ ID NO:2 and SEQ ID NO:4 is also provided. A process for determining whether a sample contains tumor cells is provided.

### BACKGROUND OF THE INVENTION

Breast cancer is a major health problem since every eighth woman in Europe and the US succumbs to this disease (Moustafa, A. S., and Nicolson, G. L., Oncol. Res. 9 (1997) 505-525; Nicolson, G. L., Biochem. Soc. Symp. 63 (1998) 231-242). Treatment includes surgery, radiation, chemotherapy and combinations thereof, depending on the stage of the disease (Schwirzke, M., et al., Anticancer Res. 19 (1999) 1801-1814). A pronounced tropism of metastasis to the bones is characteristic for this disease starting with micrometastic lesions in the bone marrow which finally may outgrow to full-blown metastases. Bone metastasis result in bone fractures and spinal cord depression syndrome often followed by severe pain, aberrant calcium homeostasis and finally lead to death of the patients (Coleman, R. E., and Rubens, R. D., Br. J. Cancer 55 (1987) 61-66).

Analysis of genes involved in breast cancer and in cancer in general revealed a dichotomy with one category of genes with deregulated expression due to mutation and the other category of genes exhibiting changes in their regulation. These findings resulted in the grouping of cancer genes into two classes: class I genes are mutated or deleted, class II genes exhibit no alterations at the DNA level (Sager, R., Science 246 (1989) 1406-1412; Sager, R., Proc. Natl. Acad. Sci. USA 94 (1997) 952-955).

### SUMMARY OF THE INVENTION

In accordance with the present invention, a protein and the related gene, termed PKW, is provided which is upregulated in tumor cells, preferably in mammary tumor cells, as compared to their non-tumor counterparts. The PKW gene codes preferably for a polypeptide consisting of SEQ ID NO:2 or SEQ ID NO:4.

The present invention provides a nucleic acid which is upregulated in tumor cells, especially in mammary carcinoma cells, and which codes for a polypeptide which induces tumor progression or metastasis, the nucleic acid being selected from the group consisting of:

(a) SEQ ID NO: 1;

(b) a nucleic acid sequence which hybridizes under stringent conditions with a nucleic acid probe of the complementary sequence of (a);

(c) a nucleic acid sequence which, because of the degeneracy of the genetic code, is not a sequence of (a) or (b), but which codes for a polypeptide having exactly the same amino acid sequence as a polypeptide encoded by a sequence of (a) or (b); and

(d) a nucleic acid sequence which is a fragment of any of the sequences of (a), (b) or (c),

with the proviso that said nucleic acid sequence is not identical to SEQ ID NO:12 or to the complementary sequence.

Preferably, the nucleic acid encodes a polypeptide consisting of amino acids of SEQ ID NO:2 or SEQ ID NO:4 and having preferably a length of at least 138 nucleotides.

The present invention further provides a purified polypeptide having a sequence of amino acids SEQ ID NO:2 or SEQ ID NO:4.

The present invention further provides a process for detecting the presence or absence of at least one specific nucleic acid or mixture of nucleic acids, or distinguishing between two different sequences in a sample, wherein said sample is suspected of containing said sequence or sequences and is preferably derived from a patient who is suffering from cancer or who is suspected of having cancer, which process comprises the following steps:

(a) incubating said sample under stringent hybridization conditions with a nucleic acid probe which is selected from the group consisting of:

(i) a nucleic acid sequence of SEQ ID NO:1 or a fragment thereof;

(ii) a nucleic acid sequence which is complementary to any nucleic acid sequence of (i);

(iii) a nucleic acid sequence which hybridizes under stringent conditions with the sequence of (i); and

(iv) a nucleic acid sequence which hybridizes under stringent conditions with the sequence of (ii); and

(b) determining whether said hybridization has occurred.

Preferably, said sequence is not identical to SEQ ID NO:12 and/or has a length of at least 138 nucleotides.

Moreover, the present invention provides a process for determining whether or not a test sample of tissue or fluid of a patient contains tumor cells or is derived from tumor cells, wherein the test sample and a second sample originating from non-tumor cells from the same individual or a different individual of the same species are used, which process comprises the following steps:

(a) incubating each respective sample under stringent hybridization conditions with a nucleic acid probe which is selected from the group consisting of:

(i) a nucleic acid sequence of SEQ ID NO: 1, or a fragment thereof;

(ii) a nucleic acid sequence which is complementary to any nucleic acid sequence of (i);

(iii) a nucleic acid sequence which hybridizes under stringent conditions with the sequence of (i); and

(iv) a nucleic acid sequence which hybridizes under stringent conditions with the sequence of (ii); and

(b) determining the approximate amount of hybridization of each respective sample with said probe, and

(c) comparing the approximate amount of hybridization of the test sample to an approximate amount of hybridization of said second sample to identify whether or not the test sample contains a greater amount of the specific nucleic acid or mixture of nucleic acids than does said second sample.

The invention further provides a method for the detection of mammary carcinoma as PKW is expressed only in mammary carcinoma cells or in metastatic cells thereof.

### BRIEF DESCRIPTION OF THE FIGURES

FIG. 1 Northern blot revealing differentially expressed mRNA's in cell lines derived from normal human mammary gland and from breast carcinomas of distinct stages of progression.

RNA was extracted from confluent cell lines, separated on a denaturing 1% agarose-formaldehyde gel, transferred to a positively charged nylon membrane and hybridized to an [α-³²P]-labeled probe corresponding to the appropriate subcloned fragment as revealed by the Differential Display Technique.

Lane a: HMEC, normal Human Mammary Epithelial Cells; lane b: cell line AR derived from medullary mammary carcinoma; lane c: cell line WA derived from invasive ductal mammary carcinoma; lanes d, e and f: cell lines 1590, HG15 and KM22, derived from mammary carcinoma bone marrow micrometastases; lane g: metastatic mammary carcinoma cell line KS, derived from malignant ascites fluid.

FIG. 2 Schematic outline of transcripts of gene PKW as well as potential proteins encoded by these transcripts. Corresponding regions and domains are highlighted by conserved symbols.

FIG. 3 Multiple Tissue Array Normalized poly A+ RNA from different tissues and cell lines were hybridized to a 32P-labeled probe derived from gene PKW. E6 corresponds to 1 μg and H6 to 0.1 μg poly A⁺ RNA from cell line AR. The code is revealed below.

FIG. 4 Detection of transcripts of gene PKW in mammary carcinomas by RT-PCR. RNA was extracted from mammary carcinomas and analyzed for transcripts corresponding specifically to the small transcript of gene PKW as described in Example 9. Lane 0: DNA Molecular Weight Marker XIV (Roche Diagnostics GmbH, DE); I: cell line AR; II-IX: different mammary carcinomas samples; lane a: β actin control (2.5 μg RNA+specific primers for β actin); lane b: (2.5 μg RNA+specific primers for gene PKW); lane c: negative controls without RT:2.5 μg RNA+specific primers for gene PKW.

An aliquot (15 μl) of the PCR products was analyzed on a 1.5% agarose gel. The bands specifically corresponding to mRNAs for gene PKW (137 bp) and β-actin (587 bp) are depicted by arrows.

### DETAILED DESCRIPTION OF THE INVENTION

The present invention provides the new gene PKW, proteins coded thereby, and use of the PKW gene for diagnostics and therapeutics, especially in the field of cancer. In particular, the invention involves the identification of said gene PKW in tumor cells, especially in mammary carcinoma cells and tumor-cell-derived material such as DNA and RNA extracts from cells. The invention also relates to the detection of tumor cells and to gene therapy methods to modulate or inhibit PKW in its function in tumor cells.

The invention comprises a nucleic acid (PKW) which has upregulated expression in tumor cells and which is capable of inducing tumor progression and/or metastasis, especially in mammary carcinoma cells. The nucleic acid (PKW) has the sequence SEQ ID NO:1 or is a nucleic acid which, because of the degeneracy of the genetic code, differs from SEQ ID NO:1 and is preferably a nucleic acid which encodes the amino acid sequence of SEQ ID NO:2 or SEQ ID NO:4.

The invention further comprises recombinant polypeptides which are coded by the nucleic acid sequences according to the invention, preferably by the DNA sequence shown in SEQ ID NO:1 or fragments thereof preferably encoding the polypeptides of SEQ ID NO:2 or SEQ ID NO:4.

The PKW polypeptide can occur in natural allelic variations which differ from individual to individual. Such variations of the amino acids are usually amino acid substitutions. However, they may also be deletions, insertions or additions of amino acids to the total sequence. The PKW polypeptide according to the invention—depending, both in respect of the extent and type, on the cell and cell type in which it is expressed—can be in glycosylated or non-glycosylated form. Polypeptides according to the invention can be identified by transfection of PKW-negative non-tumor cells with expression vectors for PKW, establishment of stable transfectants and evaluation of their tumor progression capacity after xenografting into nude mice.

“Polypeptide with PKW activity or PKW” means also a protein with minor amino acid variations but with substantially the same PKW activity. “Substantially the same” means that the activities are of the same biological properties and the polypeptides show at least 90% homology (identity) in amino acid sequence.

The term “nucleic acid molecule or nucleic acid” denotes a polynucleotide molecule which can be, for example, a DNA, RNA, or derivatized active DNA or RNA. DNA and/or RNA molecules are preferred, however.

The term “hybridize under stringent conditions” means that two nucleic acid fragments are capable of hybridization to one another under standard hybridization conditions described in Sambrook et al., Molecular Cloning: A Laboratory Manual (1989) Cold Spring Harbor Laboratory Press, New York, USA. More specifically, “stringent conditions” as used herein refer to hybridization in 6.0×SSC at 45° C. to 55° C., preferably 50° C. to 55° C., followed by a wash. This wash can be with 2.0×SSC at 50° C. Preferably, hybridization is performed using the commercially available Express Hyb™ Hybridization Solution of Clontech, which is a non-viscious solution containing no salmon sperm DNA. The stringency of the salt concentration in the wash step can be selected, for example, from about 2.0×SSC at 50° C., for low stringency, to about 0.2×SSC at 50° C., for high stringency. In addition, the temperature in the wash step can be increased from low stringency conditions at room temperatures, about 22° C., to high stringency conditions at about 65° C.

The phrase “nucleic acid or polypeptide” as used throughout this application refers to a nucleic acid or polypeptide having a PKW activity which is substantially free of cellular material or culture medium when produced by recombinant DNA techniques, or substantially free of chemical precursors or other chemicals when synthesized chemically. Such a nucleic acid is preferably free of sequences which naturally flank the nucleic acid (i.e. sequences located at the 5′ and the 3′ ends of the nucleic acid) in the organism from which the nucleic acid is derived.

The polypeptides according to the invention can be produced by recombinant means, or synthetically. Non-glycosylated PKW polypeptide is obtained when it is produced recombinantly in prokaryotes. With the aid of the nucleic acid sequences provided by the invention it is possible to search for the PKW gene or its variants in genomes of any desired cells (e.g. apart from human cells, also in cells of other mammals), to identify these and to isolate the desired gene coding for the PKW proteins. Such processes and suitable hybridization conditions are known to a person skilled in the art and are described, for example, by Sambrook et al., Molecular Cloning: A Laboratory Manual (1989) Cold Spring Harbor Laboratory Press, New York, USA, and Hames, B. D., Higgins, S. G., Nucleic Acid Hybridisation—A Practical Approach (1985) IRL Press, Oxford, England. In this case the standard protocols described in these publications are usually used for the experiments.

With the aid of such nucleic acids coding for a PKW polypeptide, the polypeptide according to the invention can be obtained in a reproducible manner and in large amounts. For expression in prokaryotic or eukaryotic organisms, such as prokaryotic host cells or eukaryotic host cells, the nucleic acid is integrated into suitable expression vectors, according to methods familiar to a person skilled in the art. Such an expression vector preferably contains a regulatable/inducible promoter. These recombinant vectors are then introduced for the expression into suitable host cells such as, e.g., E. coli as a prokaryotic host cell or Saccharomyces cerevisiae, Teratocarcinoma cell line PA-1, sc 9117 (Büttner et al., Mol. Cell. Biol. 11 (1991) 3573-3583), insect cells, CHO or COS cells as eukaryotic host cells and the transformed or transduced host cells are cultured under conditions which allow expression of the heterologous gene. The isolation of the protein can be carried out according to known methods from the host cell or from the culture supernatant of the host cell. Such methods are described for example by Ausubel I., Frederick M., Current Protocols in Mol. Biol. (1992), John Wiley and Sons, New York. Also in vitro reactivation of the protein may be necessary if it is not found in soluble form in the cell culture.

PKW polypeptide can be purified after recombinant production by affinity chromatography using known protein purification techniques, including immunoprecipitation, gel filtration, ion exchange chromatography, chromatofocussing, isoelectric focussing, selective precipitation, electrophoresis, or the like.

The invention further comprises recombinant expression vectors which are suitable for the expression of PKW, recombinant host cells transfected with such expression vectors, as well as a process for the recombinant production of a protein which is encoded by the PKW gene.

The invention further comprises a method for detecting a nucleic acid molecule of gene PKW, comprising incubating a sample (e.g., body fluids such as blood, cell lysates or a reverse transcript of an RNA sample) with the nucleic acid molecule according to the invention and determining hybridization under stringent conditions of said nucleic acid molecule to a target nucleic acid molecule for determination of presence of a nucleic acid molecule which is the PKW gene and therefore a method for the identification of tumor cells and preferably of mammary tumors. Quantitative detection can be performed by PCR techniques, preferably by the use of quantitative RT-PCR using, e.g., the LightCycler® of Roche Diagnostics GmbH, DE.

To determine whether a test sample contains tumor cells, the approximate amount of hybridization of the nucleic acid with the target nucleic acid or nucleic acids is determined. The approximate amount of hybridization need not be determined quantitatively, although a quantitative determination is encompassed by the present invention. Typically, the approximate amount of hybridization is determined qualitatively, for example, by a sight inspection upon detecting hybridization. For example, if a gel is used to resolve labelled nucleic acid which hybridizes to target nucleic acid in the sample, the resulting band can be inspected visually. When performing a hybridization of isolated nucleic acid which is free from tumor cells from an individual of the same species, the same protocol is followed. One can compare the approximate amount of hybridization in the test sample to the approximate amount of hybridization in the sample free from tumor cells, to identify whether or not the test sample contains a greater amount of the target nucleic acid or nucleic acids than does the sample which is free from tumor cells.

In a further method according to the invention no second sample is used. For the detection whether the expression of PKW gene is upregulated, the level of mRNA of PKW is compared with the level of mRNA of a standard gene (housekeeping gene (see, e.g., Shaper, N. L., et al., J. Mammary Gland Biol. Neoplasia 3 (1998) 315-324; Wu, Y. Y., et al., Acta Derm. Venerol. 80 (2000) 2-3) of the cell, preferably by RT-PCR.

For visual inspection in particular, it is recommended that an appreciable difference by visualized to assess that the test sample contains a greater amount of the target nucleic acid or nucleic acids.

As is shown in accordance with the present invention, the PKW nucleic acid is expressed in a greater amount in a tumor sample than in a sample free from tumor cells and/or in a greater amount than a housekeeping gene. A test sample containing tumor cells will have a greater amount of the PKW nucleic acid than does a sample which is free from tumor cells. To identify a test sample as containing upregulated PKW nucleic acid, i.e., wherein the cells are tumor cells or are tumor cells of a mammary carcinoma, it is preferable that the test sample have an approximate amount of PKW nucleic acid which is appreciably greater that the approximate amount in a sample free of tumor cells. For example, a test sample having an upregulated PKW gene may have approximately 15- to approximately 60-fold greater amount of PKW gene than a sample free of tumor cells or an at least 3-fold greater amount of PKW mRNA than mRNA of a housekeeping gene like glycerolaldehyde-3-phosphate dehydrogenase (GPDH) or porphobilinogen deaminase.

On the basis of the nucleic acids provided by the invention it is possible to provide a test which can be used to detect nucleic acids with upregulated expression in human tumor cells. Such a test can be carried out by means of nucleic acid diagnostics. In this case the sample to be examined is contacted with a probe that is selected from the group comprising

a) the nucleic acid sequence shown in SEQ ID NO:1, fragments thereof or a nucleic acid sequence which is complementary to one of these nucleic acid sequences, and

b) nucleic acids which hybridize under stringent conditions with one of the nucleic acids from a), wherein

the nucleic acid probe is incubated with the nucleic acid of the sample and the hybridization is detected optionally by means of a further binding partner for the nucleic acid of the sample and/or the nucleic acid probe. For obtaining a nucleic acid by hybridization in accordance with b), it is preferable to hybridize to a probe selected from the group of nucleic acids defined by the bases 724 to 1235 (small transcript) or bases 1831-2342 (large transcript) of SEQ ID NO:1, a fragment thereof of at least 200 bases or a sequence complementary thereto. Hybridization between the probe used and nucleic acids from the sample indicates the presence of the RNA of such proteins.

Methods of hybridization of a probe and a nucleic acid are known to a person skilled in the art and are described, for example, in WO 89/06698, EP-A 0 200 362, U.S. Pat. No. 2,915,082, EP-A 0 063 879, EP-A 0 173 251, EP-A 0 128 018.

In a preferred embodiment of the invention the coding nucleic acid of the sample is amplified before the test, for example by means of the known PCR technique. Usually a derivatized (labeled) nucleic acid probe is used within the framework of nucleic acid diagnostics. This probe is contacted with a denatured DNA, RNA or RT-DNA from the sample which is bound to a carrier and in this process the temperature, ionic strength, pH and other buffer conditions are selected—depending on the length and composition of the nucleic acid probe and the resulting melting temperature of the expected hybrid—such that the labeled DNA or RNA can bind to homologous DNA or RNA (hybridization see also Wahl, G. M., et al., Proc. Natl. Acad. Sci. USA 76 (1979) 3683-3687). Suitable carriers are membranes or carrier materials based on nitrocellulose (e.g., Schleicher and Schüll, BA 85, Amersham Hybond, C.), strengthened or bound nitrocellulose in powder form or nylon membranes derivatized with various functional groups (e.g., nitro groups) (e.g., Schleicher and Schüll, Nytran; NEN, Gene Screen; Amersham Hybond M.; Pall Biodyne).

Hybridizing DNA or RNA is then detected by incubating the carrier with an antibody or antibody fragment after thorough washing and saturation to prevent unspecific binding. The antibody or the antibody fragment is directed towards the substance incorporated during hybridization to the nucleic acid probe. The antibody is in turn labeled. However, it is also possible to use a directly labeled DNA. After incubation with the antibodies it is washed again in order to only detect specifically bound antibody conjugates. The determination is then carried out according to known methods by means of the label on the antibody or the antibody fragment.

The detection of the expression can be carried out for example as:

in situ hybridization with fixed whole cells, with fixed tissue smears,

colony hybridization (cells) and plaque hybridization (phages and viruses),

Southern hybridization (DNA detection),

Northern hybridization (RNA detection),

serum analysis (e.g., cell type analysis of cells in the serum by slot-blot analysis),

after amplification (e.g., PCR technique).

Therefore the invention also includes a method for the detection of carcinoma cells, comprising

a) incubating a sample of a patient suffering from cancer, selected from the group of body fluid, of cells, or of a cell extract or cell culture supernatants of said cells, whereby said sample contains nucleic acids with a nucleic acid probe which is selected from the group consisting of

(i) the nucleic acid shown in SEQ ID NO:1 or a nucleic acid which is complementary to said sequence, and

(ii) nucleic acids which hybridize with one of the nucleic acids from (i) and detecting hybridization, preferably by means of a further binding partner of the nucleic acid of the sample and/or the nucleic acid probe or by X-ray radiography.

In addition, the invention comprises a process for determining whether or not a test sample originating from or containing human cells has a tumor progression potential, which process comprises the following steps:

(a) incubating a first compartment of said sample under stringent hybridization conditions with a first nucleic acid probe which is selected from the group consisting of:

(i) a nucleic acid with a sequence of SEQ ID NO:1 or a fragment thereof;

(ii) a nucleic acid with a sequence which is complementary to any nucleic acid of (i);

(iii) a nucleic acid with a sequence which hybridizes under stringent conditions with the nucleic acid of (i); and

(iv) a nucleic acid with a sequence which hybridizes under stringent conditions with the nucleic acid of (ii); and

(b) incubating a second compartment of said sample under stringent hybridization conditions with a second nucleic acid probe being a housekeeping gene or a fragment thereof;

(c) determining the approximate amount of hybridization of said sample with said first and second probe;

(d) identifying whether or not the test sample contains an at least 3-fold amount of nucleic acid hybridizing with the first probe in comparison to the amount of nucleic acid hybridizing with the second probe.

Preferably, the nucleic acid probe is incubated with the nucleic acid of the sample and the hybridization is detected optionally by means of a further binding partner for the nucleic acid of the sample and/or the nucleic acid probe. As probes, nucleic acids selected from the group consisting of nucleid acids defined by the bases 724 to 1235 (small transcript) or bases 1831-2342 (large transcript) of SEQ ID NO:1, a fragment thereof of at least 200 bases or a sequence complementary thereto are preferred.

The nucleic acids according to the invention are hence valuable markers in the diagnosis and characterization of tumors, especially of mammary tumors.

The invention further comprises a method for producing a protein whose expression is correlated with tumors, by expressing an exogenous DNA in prokaryotic or eukaryotic host cells and isolation of the desired protein, wherein the protein is coded by the nucleic acid molecules according to the invention, preferably by the DNA sequence shown in SEQ ID NO:1.

The protein can be isolated from the cells or the culture supernatant and purified by chromatographic means, preferably by ion exchange chromatography, affinity chromatography and/or reverse phase HPLC.

The invention further comprises a protein according to the invention which is encoded by a nucleic acid molecule according to the invention, preferably having the nucleotide sequence set forth in SEQ ID NO:1.

The present invention relates to the cloning and characterization of the gene PKW, which is especially characterized as a tumor progression gene, and as an upregulated gene indicative for the tumor progression potential of tumor cells, preferably of mammary tumor cells.

According to the invention inhibitors for the expression of PKW (e.g., antisense nucleotides) can be used to inhibit tumor progression, preferably of mammary carcinomas, in vivo.

The invention further provides methods for identifying and isolation of antagonists of PKW or inhibitors for the expression of PKW (e.g. antisense nucleotides). Such antagonists or inhibitors can be used to inhibit tumor progression and cause massive apoptosis of tumor cells in vivo.

According to the invention there are provided methods for identifying and isolation of PKW antagonists which have utility in the treatment of cancer. These methods include methods for modulating the expression of the polypeptides according to the invention, methods for identifying PKW antagonists which can selectively bind to the proteins according to the invention, and methods of identifying PKW antagonists which can modulate the activity of said polypeptides. The methods further include methods for modulating, preferably inhibiting, the transcription of PKW gene to mRNA. These methods can be conducted in vitro or in vivo and may make use of and establish cell lines and transgenic animal models of the invention.

A PKW antagonist is defined as a substance or compound which decreases or inhibits the biological activity of PKW, a polypeptide and/or inhibits the transcription or translation of PKW gene. In general, screening procedures for PKW antagonists involve contacting candidate substances with host cells in which invasiveness is mediated by expression of PKW under conditions favorable for measuring PKW activity.

PKW activity may be measured in several ways. Typically, the activation is apparent by a change in cell physiology, such as increased mobility and invasiveness in vitro, or by a change in the differentiation state, or by a change in cell metabolism leading to an increase of proliferation.

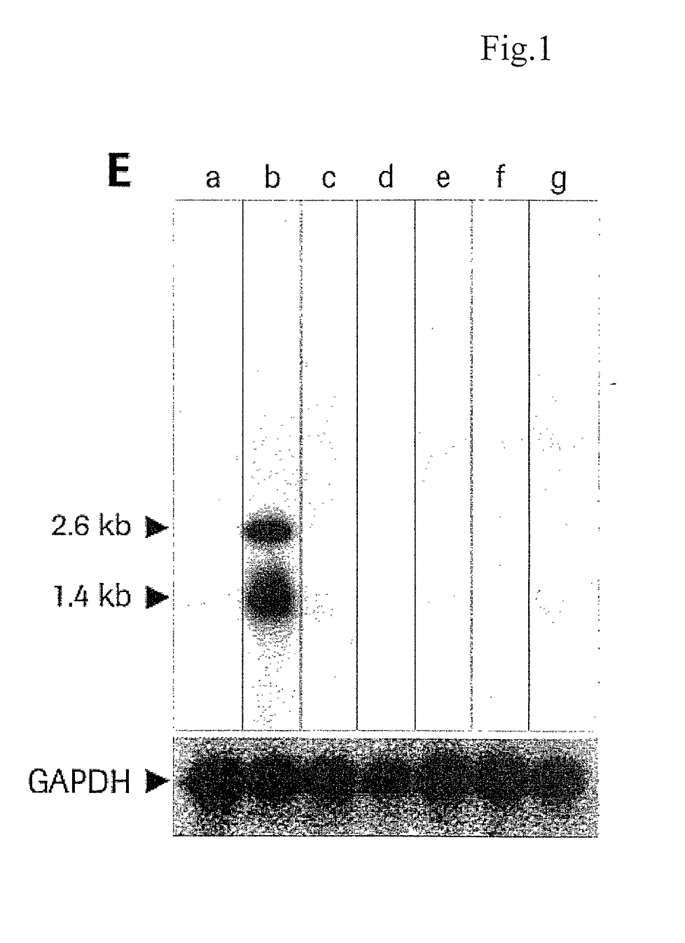

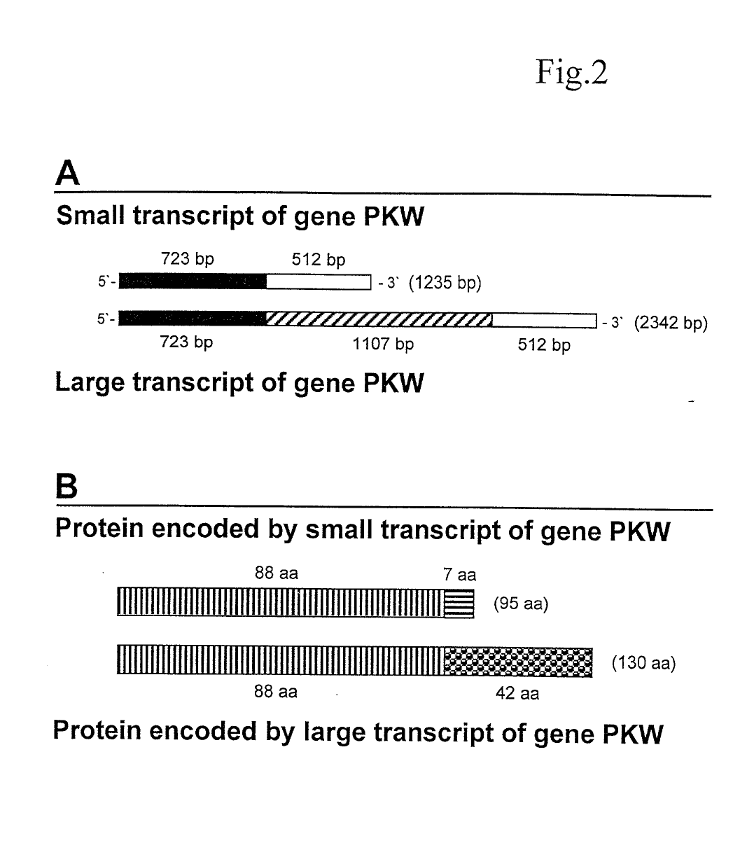

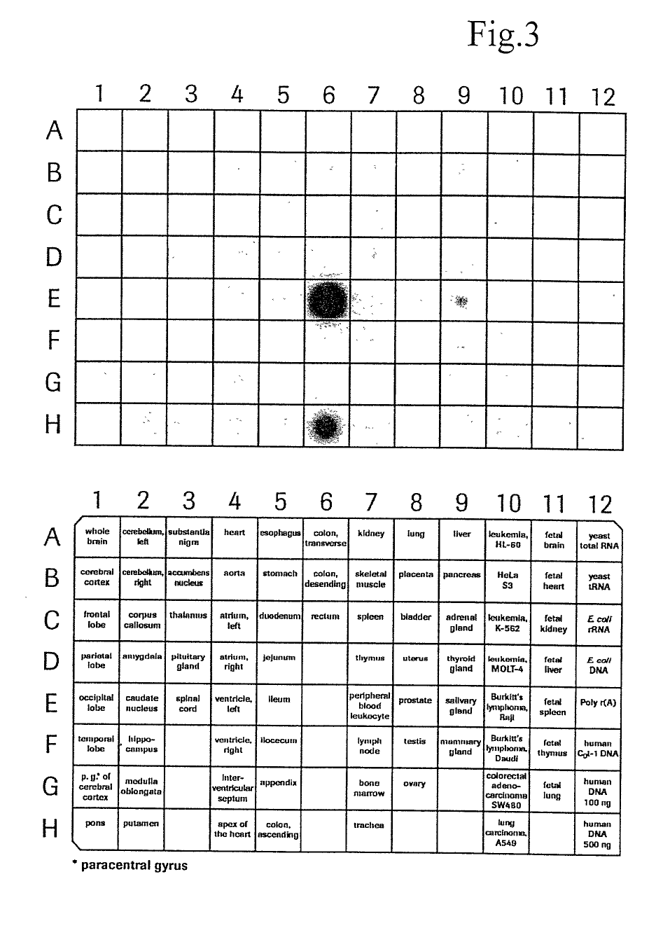

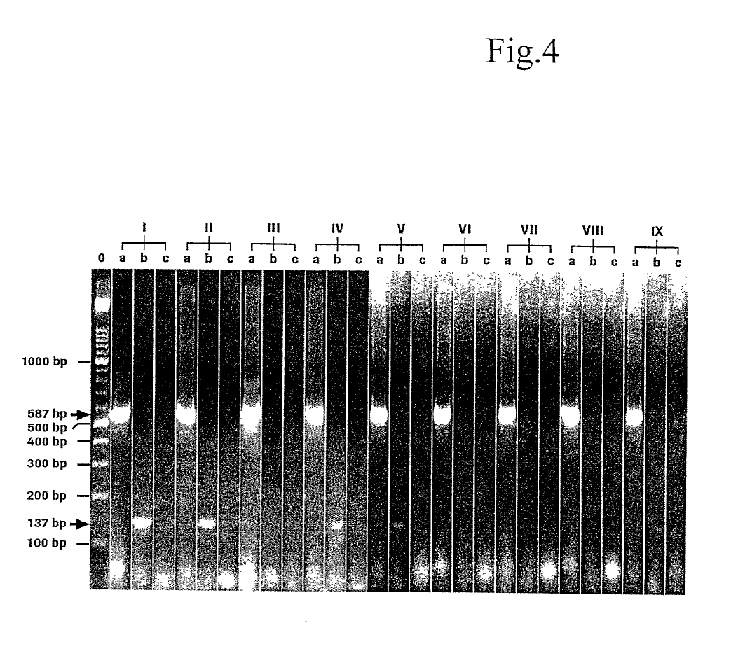

As shown in FIG. 1, gene PKW is expressed only in one of the primary carcinoma cell lines, the one derived from the medullary mammary carcinoma. Topology of small and large transcripts of gene PKW as well as the potential proteins encoded by them are outlined schematically in FIG. 2. Small and large transcript of gene PKW share 723 bp at the 5′ end and 512 bp at the 3′ end. The large transcript contains an insertion of 1107 bp (FIG. 2A). The small transcript (bp 459-723 and 1831-1850 of SEQ ID NO:1) is due to differential splicing of the large transcript. The small transcript encodes a potential protein of 95 aa, the large transcript exhibits an open reading frame of 130 aa. Both potential proteins share an open reading frame of 88 aa with exception of one different aa at position 43 (nucleic acid position 586 of SEQ ID NO:1), followed by extensions of 7 aa and 42 aa for the smaller and the larger protein in different reading frames. The difference at position 43 may be a PCR artefact or could be caused by polymorphism. The 95 aa protein exhibits an isoelectric point (IEP) of 11.2, the IEP of the 130 aa protein is 10.4. These findings point to a nuclear localization of the proteins encoded by gene PKW. A search revealed partial similarity with a clone derived from a human BAC library (AQ548392) (SEQ ID NO:12). However, the database does not provide any hints as to coding sequences and/or usefulness.

As shown in FIG. 3 transcripts of gene PKW were detected only in salivary gland, not in other adult human tissues and in a small panel of embryonic tissues such as fetal brain, heart, kidney, liver, spleen, thymus and lung. Promyelocytic leukemia cell line HL-60, HeLa cells, chronic myelogenous leukemia cell line K-562, lymphoblastic leukemia cell line MOLT-4, Burkitt lymphoma cell line Raji, colorectal adenocarcinoma cell line SW 480, lung carcinoma cell line A549 and melanoma cell line G361 all scored negative with respect to mRNA for gene PKW (FIG. 3). The probe used for hybridization detects the small as well as the large transcript of gene PKW. A panel of mammary carcinoma cell lines described in (Schwirzke, M., et al., Anticancer Res. 18 (1998) 1409-1421) also tested negative with respect to the mRNA of gene PKW by Northern blotting as well as RT-PCR. These include MDA-435 (Cailleau, R., et al., In Vitro 14 (1978) 911-915) derived subclones 4C4 and 2A5, cell lines MDA-MB231 (Cailleau, R., et al., J. Natl. Cancer Inst. 53 (1974) 661-674), MDA-MB436 (Cailleau, R., et al., In Vitro 14 (1978) 911-915), ZR-75 (Engel, L. W., et al., Cancer Res. 38 (1978) 3352-3364), T47D (Freake, H. C., et al., Biochem. Biophys. Res. Commun. 101 (1981) 1131-1138), Hs578 T (Hackett, A. J., J. Natl. Cancer Inst. 58 (1977) 1795-1806), MCF-7 (Schiemann, S., et al., Anticancer Res. 17 (1997) 13-20; Schiemann, S., et al., Clin. Exp. Metastasis 16 (1998) 129-139), MCF-7_{ADR} (Schiemann, S., et al., Anticancer Res. 17 (1997) 13-20; Lee, J. H., et al., Biochem. Biophys. Res. Commun. 238 (1997) 462-467), LCC-1, LCC-2 and LCC-9 (Brünner, N., et al., Cancer Res. 53 (1993) 283-290; Brünner, N., et al., Cancer Res. 53 (1993) 3229-3232). In summary 11 mammary carcinomas were analyzed for expression of the small transcript of gene PKW by RT-PCR. 4 carcinomas scored positive. Parts of the results are displayed in FIG. 4. Two of the positive carcinomas corresponded to ductal carcinomas and the other two matched with lobular carcinomas.

The following examples, references, sequence listing and figures are provided to aid the understanding of the present invention. It is understood that modifications can be made in the procedures set forth without departing from the spirit of the invention.

| | |
|---|---|
| SEQ ID NO:1: | cDNA of PKW gene and amino acid sequence of large |
| | PKW splice variant. |
| SEQ ID NO:2: | Amino acid of large PKW splice variant. |
| SEQ ID NO:3: | cDNA and amino acid sequence of small PKW splice |
| | variant |
| SEQ ID NO:4: | Amino acid of small PKW splice variant. |
| SEQ ID NO:5: | Primer GSP1 |
| SEQ ID NO:6: | Primer GSP2 |
| SEQ ID NO:7: | Primer AUAP |
| SEQ ID NO:8: | Primer RTR-5 |
| SEQ ID NO:9: | Primer RTF-6 |
| SEQ ID NO:10: | β-actin reverse primer |
| SEQ ID NO:11: | β-actin forward primer |
| SEQ ID NO:12: | Fragment of sequence AQ 548392 |

### EXAMPLE 1

### Cell Lines and Cell Culture

Human mammary epithelial cells (HMEC) were obtained from Bio Whittaker, Heidelberg, Germany. Primary mammary carcinoma cell lines AR and WA were obtained by fragmenting the primary tumor with scissors, treatment with collagenase (0.2 mg/ml in 5% FCS) and finally the cellular fraction was isolated by Ficoll gradient technique. Tumor cells were selected with a monoclonal antibody directed against MUC-1 coated to Dynabeads®(Dynal, Norway). MUC-1 antibodies are described in WO 99/40881. 5×10⁷ beads were mixed with 10⁷ cells, incubated for 1 h at 4° C. on a roller device, the bead fraction was collected on a magnet, washed twice with DMEM and finally the cells were propagated in 75 cm² culture flasks in DMEM supplemented with 10% FCS. Two clones were identified after 4 weeks for both cell lines referred to as AR and WA. Cell line AR is derived from an invasive medullary mammary carcinoma, cell line WA is derived from an invasive ductal carcinoma. Cell lines 1590, HG15 and KM22 are derived from bone marrow micrometastases of mammary carcinoma patients. Cellular fraction was isolated on a Ficoll gradient, erythrocytes were lysed and the cells were suspended in DMEM+10% FCS and tumor cells were isolated on Dynabeads® coupled with MUC-1 antibody as described above. The bead fraction was cultivated in DMEM+10% FCS, 10 μg/ml insulin and 10 μg/ml transferrin. Outgrowth of tumor cell lines was observed after 8 weeks. Clones were isolated by treatment with EDTA and propagated under standard conditions as described above. Cell line KS was isolated from malignant ascites fluid of a mammary carcinoma patient. 2000 ml of ascites were collected and the cellular fraction was isolated on a Ficoll gradient. 2×10⁷ cells were seeded into 750 cm² culture flasks. Tumor cell clusters were obtained from the culture supernatants in order to separate them from adherently growing fibroblasts and mesothelial cells (passages 1-4). Passages 5-10 resulted in cultures growing partly as a monolayer and in suspension. Finally cells were propagated as a monolayer in DMEM+10% FCS.

### EXAMPLE 2

### mRNA Differential Display PCR

Differential display reverse transcriptase polymerase chain reaction (DD-RT-PCR) was performed following the method described by Liang and Pardee using the RNAimage™ kits (GenHunter Corp. Brookline, Mass.) according to the manufacturer's protocol.

Total RNA was isolated from frozen cell pellets of all cell lines listed above making use of the RNeasy Midi® Kit (Qiagen, www.qiagen.de). Chromosomal DNA was removed from RNA samples by digestion at 37° C. for 30 min with RNAse-free DNAse I using the MessageClean Kit® (GenHunter Corp. Brookline, Mass.).

RNA was used as a template for first strand cDNA synthesis in the presence of 3 different one-base anchored oligo-dT primers (H-T₁₁M, where M maybe G, A or C).

For a 20 μl reaction, 1 μl of DEPC-treated H₂O, 4 μl of 5× reverse transcriptase buffer [125 mM Tris-Cl, pH 8.3, 188 mM KCl, 7.5 mM MgCl₂, 25 mM dithiothreitol (DDT)], 10 μl of dNTP mix [250 μM each], 2 μl of H-T₁₁M primer [2 μM], and 2 μl of DNA-free total RNA sample [0.1 μg/μl] were mixed. The solution was heated to 65° C. for 5 min and cooled to 37° C. for 10 min, and 1 μl [100 units] of Moloney murine leukemia virus (MMLV) reverse transcriptase was added. After incubation at 37° C. for 1 h, the reaction was terminated by incubation at 75° C. for 5 min. The following PCR procedure was performed in a 20 μl reaction, containing 2 μl of reverse transcription reaction mixture, 9.2 μl of DEPC-treated H₂O, 2 μl pf 10× PCR buffer [100 mM Tris-Cl, pH 8.4, 500 mM KCl, 15 mM MgCl₂, 0.01% gelatine], 1.6 μl of dNTP mix [25 μM each], 2 μl of the respective H-T₁₁M primer [2 μM], 2 μl of an arbitrary 13-mer primer, [2 μM], 1 μl of α-[³⁵S]dATP [>1000 Ci/mmole] and 0.2 μl of AmpliTag [10 units/μl] DNA polymerase (Perkin Elmer, Norwalk, Conn.). PCR included a total of 40 cycles at 94° C. for 30 s, 40° C. for 2 min, 72° C. for 30 s, and finally 5 min at 72° C. After adding 2 μl loading buffer to 3.5 μl of each sample, the PCR products of all cell lines were heated at 80° C. for 2 min and loaded in parallel on a denaturing 6% polyacrylamide sequencing gel for electrophoresis. The dried gel was exposed to BioMax™ MR film (Kodak) at room temperature for 24 to 48 h and the autoradiogram was analyzed for the differentially expressed genes. Bands corresponding to cDNA's of interest reproducibly displayed in two independent DD-RT-PCR reactions were excised from the dried gel, and the cDNA was eluted from the gel by soaking the gel slice in 100 μl of dH₂O of 10 min and boiled for 15 min. After addition of 10 μl of 3M NaOAc and 5 μl glycogen [10 mg/ml] as carrier the cDNA-fragments were recovered by precipitation with 450 μl of ethanol and redissolved in 10 μl dH₂O. 4 μl eluted cDNA was reamplified in a second PCR using the same primer set and conditions except the dNTP concentrations of 20 μM each and no radioistope. As a control, gel slices were excised from lanes without visible bands on a level with the detected cDNA fragments of interest and treated as described above. The amplified PCR products obtained were analyzed on a 3% NuSieve® GTG (FMC BioProducts, Rockland), agarose gel, then purified using the QIAquick™ Gel Extraction kit (Qiagen, DE) and used as probes for Northern analysis.

### EXAMPLE 3

### DNA Sequencing of DD-RT-PCR Fragments

All PCR fragments of interest were sequenced directly after extraction and purification from agarose gels. The nucleotide sequences data were analyzed for homologies with known genes or EST's in the current DNA data bases.

### EXAMPLE 4

### Northern Blot Analysis

Poly A⁺-RNA was isolated from total RNA. 1 μg of poly⁺-RNA from HMEC, AR, WA, 1590, KM22, HG15 and KS cells were loaded side by side on a denaturing 1% agarose formaldehyde gel and then size-separated by electrophoresis. Blotting to positively charged nylon membrane was done by capillary downward transfer. After UV-crosslinking (Stratagene UV Stratalinker™ 2400, www.stratagene.com) blots were hybridized. For that the DD-RT-PCR products were labeled with a α-[³²P]dATP up to a specific activity of 2×10⁹ cpm/μg. Prehybridization (30 min) and hybridization (over-night) with radioactive probes were performed in ExpressHyb™ hybridization solution (Clontech, www.clontech.com) at 68° C. according to the manufacturer's recommendation. Membranes were washed in solution 1 (2× SSC, 0.05% SDS) at room temperature for 30-40 min with continuous agitation and several replacements of the wash solution 1 followed by a washing step with solution 2 (0.1× SSC, 0.1% SDS) at 50° C. for 40 min with one change of fresh solution. The membranes were then exposed to Cronex™, Medical X-Ray Films (Sterling Diagnostic Imaging Inc., USA) at −80° C. for 3 to 72 h. Equal loading and transfer of mRNA to the membrane was assessed by rehybridizing the blots with β-[³²P]dATP-labeled glyceraldehyde-3-phosphate dehydrogenase (GAPDH).

### EXAMPLE 5

### Cloning of DD-RT-PCR Fragments

Northern analysis was first performed using hybridization probes generated directly by PCR reamplification. Those amplified PCR fragments corresponding to differentially expressed mRNAs on a Northern blot were subcloned Subcloned fragments were isolated and stored for further experiments to verify differential expression.

### EXAMPLE 6

### 5′ RACE PCR

This method was applied to isolate the cDNA's of gene PKW. To identify the 5′-sequences of both transcripts a 5′RACE (Rapid Amplification of cDNA Ends) PCR was performed following the manual as described in the 5′ RACE System for Rapid Amplification of cDNA Ends Kit, Version 2.0 (Gibco BRL, Life Technologies). First strand cDNA was synthesized from total RNA (without digestion with DNase I) using the gene-specific primer GSP1 (5′TTATCTTTATTCATTTTGG-3′, SEQ ID NO:5) and SuperScript™ II, an RNAse H⁻ derivative of the Moloney Murine Leukemia Virus Reverse Transcriptase (M-MVL RT). After cDNA synthesis, the solution was purified from unincorporated dNTPs and GSP1. TdT (Terminal deoxynucleotidyl transferase) was used to add homopolymeric tails to the 3′ ends of the cDNA. Tailed cDNA then was amplified by PCR using a nested, gene-specific primer GSP2(5′TGCGGGACTCGTCGTAAGTATGC-3′, SEQ ID NO:6),which anneals 3′ to GSP1, and the deoxyinosine-containing abriged universal amplification primer AUAP (5′GGCCACGCGTCGACTAGTAC-3′, SEQ ID NO:7). After several reactions with varying parameters also the longer cDNA (corresponding to the 2.6 kb transcript ) could be detected in addition to the shorter one, which appears in any reaction. The enriched and purified cDNA's were cloned and sequenced.

### EXAMPLE 7

### Human Multiple Tissue Expression Array (MTE™)

This array (Clontech, Palo Alto, Calif.) contains normalized loadings of poly A⁺-RNA from 76 different human tissues as well as control RNAs and DNAs as revealed in FIG. 5. The blot was hybridized with an α-[³²P]dATP PKW cDNA according to the instructions of the manufacturer and exposed to X-ray film at −70° C.

### EXAMPLE 8

### Isolation of RNA from Breast Tumor Tissues

Total RNA was isolated from frozen tumor samples. The frozen tissues were covered with the suggested amount of lysis buffer and immediately disrupted and homogenized by means of a homogenizer for 45-60 sec and 20000 U/min. The homogenized lysate was further processed as described in the manufacturer's protocol.

### EXAMPLE 9

### Reverse Transcription Polymerase Chain Reaction (RT-PCR)

In order to eliminate genomic DNA contamination the total RNA samples were treated with RNAse-free DNAseI at 37° C. for 30 min. First strand synthesis was performed following the protocol of the first strand cDNA Synthesis Kit for RT-PCR (Roche Diagnostics GmbH, DE) by using the specific reverse primer RTR-5 (5′CCATTCATTCATTTTCAAG3′, SEQ ID NO:8). The reverse transcription reaction was performed at 25° C. for 10 min and then at 55° C. for 60 min. After incubation, the AMV Reverse Transcriptase was denatured at 99° C. for 5 min. For each sample a negative control reaction without AMV Reverse Transcriptase was performed.

The resulting single-stranded cDNA was amplified by PCR (High Fidelty PCR Master, Roche Diagnostics GmbH, DE) utilizing a second specific forward primer RTF-6 (5′AAAACGCATGGCTTGTC3′, SEQ ID NO:9). The amplification was performed with an initial denaturation step at 94° C. for 2 min, 10 cycles of 15 s denaturation at 94° C., 30 s annealing at 57° C. and 1 min elongation at 72° C., followed by cycles under same conditions. Equal loading and integrity of mRNA was assessed by a control RT-PCR with β-actin primers (reverse primer 5′AGGGTACATGGTGGTGCCGCCAGAC3′ SEQ ID NO:10 forward primer 5′CCAAGGCCAACCGCGAGAAGATGAC3′ SEQ ID NO:11).

List of References

Ausubel I., Frederick M., Current Protocols in Mol. Biol. (1992), John Wiley and Sons, New York

Brünner, N., et al., Cancer Res. 53 (1993) 283-290

Brünner, N., et al., Cancer Res. 53 (1993) 3229-3232

Büttner et al., Mol. Cell. Biol. 11 (1991) 3573-3583

Cailleau, R., et al., In Vitro 14 (1978) 911-915

Cailleau, R., et al., J. Natl. Cancer Inst. 53 (1974) 661-674

Coleman, R. E., and Rubens, R. D., Br. J. Cancer 55 (1987) 61-66

Engel, L. W., et al., Cancer Res. 38 (1978) 3352-3364

EP-A 0 063 879

EP-A 0 128 018

EP-A 0 173 251

EP-A 0 200 362

Freake, H. C., et al., Biochem. Biophys. Res. Commun. 101 (1981) 1131-1138

Hackett, A. J., J. Natl. Cancer Inst. 58 (1977) 1795-1806

Hames, B. D., Higgins, S. G., Nucleic Acid Hybridisation—A Practical Approach (1985) IRL Press, Oxford, England

Lee, J. H., et al., Biochem. Biophys. Res. Commun. 238 (1997) 462-467

Liang, P., and Pardee, A. B., Science 257 (1992) 967-971

Liang, P., et al., Cancer Res. 52 (1992) 6966-6968

Liang, P., et al., Nucleic Acids Res. 21 (1993) 3269-3275

Moustafa, A. S., and Nicolson, G. L., Oncol. Res. 9 (1997) 505-525

Nicolson, G. L., Biochem. Soc. Symp. 63 (1998) 231-242

Sager, R., Proc. Natl. Acad. Sci. USA 94 (1997) 952-955

Sager, R., Science 246 (1989) 1406-1412

Sambrook et al., Molecular Cloning: A Laboratory Manual (1989) Cold Spring Harbor Laboratory Press, New York, USA

Schiemann, S., et al., Anticancer Res. 17 (1997) 13-20

Schiemann, S., et al., Clin. Exp. Metastasis 16 (1998) 129-139

Schwirzke, M., et al., Anticancer Res. 18 (1998) 1409-1421

Schwirzke, M., et al., Anticancer Res. 19 (1999) 1801-1814

Shaper, N. L., et al., J. Mammary Gland Biol. Neoplasia 3 (1998) 315-324

U.S. Pat. No. 2,915,082

Wahl, G. M., et al., Proc. Natl. Acad. Sci. USA 76 (1979) 3683-3687

WO 89/06698

Wu, Y. Y., et al., Acta Derm. Venerol. 80 (2000) 2-3

What is claimed is:

1. A process for determining whether or not a test sample originating from or containing human cells has a tumor progression potential, wherein a second sample originating from non-tumor cells from the same individual or a different individual of the same species is also used, which process comprises the following steps:
(a) incubating said samples under stringent hybridization conditions with a nucleic acid probe which is selected from the group consisting of:
(i) a nucleic acid with a sequence of SEQ ID NO:1 or a fragment thereof;
(ii) a nucleic acid with a sequence which is complementary to any nucleic acid of (i);
(iii) a nucleic acid with a sequence which hybridizes under stringent conditions with the nucleic acid of (i); and
(iv) a nucleic acid with a sequence which hybridizes under stringent conditions with the nucleic acid of (ii); and
(b) determining the approximate amount of hybridization of each respective sample with said probe and
(c) comparing the approximate amount of hybridization of the test sample to an approximate amount of hybridization of said second sample to identify whether or not the test sample contains a lower amount of the nucleic acid than does said second sample.

2. A process for determining whether or not a test sample originating from or containing human cells has a tumor progression potential, which process comprises the following steps:
(a) incubating a first compartment of said sample under stringent hybridization conditions with a first nucleic acid probe which is selected from the group consisting of:
(i) a nucleic acid with a sequence of SEQ ID NO:1 or a fragment thereof;
(ii) a nucleic acid with a sequence which is complementary to any nucleic acid of (i);
(iii) a nucleic acid with a sequence which hybridizes under stringent conditions with the nucleic acid of (i); and
(iv) a nucleic acid with a sequence which hybridizes under stringent conditions with the nucleic acid of (ii); and
(b) incubating a second compartment of said sample under stringent hybridization conditions with a second nucleic acid probe being a housekeeping gene or a fragment thereof;
(c) determining the approximate amount of hybridization of said sample with said first and second probe;
(d) identifying whether or not the test sample contains an at least 3-fold amount of nucleic acid hybridizing with the first probe in comparison to the amount of nucleic acid hybridizing with the second probe.


SEQUENCE LISTING (12 sequences)

SEQ ID NO: 1 - DNA, 2342 bp, Homo sapiens
  CDS (459)..(848)
aacctccaccacaacgctcacccttacagacacactattgcaggtctccgagggcctttgggaggccctgcttcctgcgagctgtcccggcaggacagagactcttcccgccgcggccctgccattccaggctgaggctgtgagcagcaccatgacaagctccggccgcagtggctctcaacagtgtgggtctctgaccacccgacgagctggaagtgcagaccgctgacctcccttgagaacctactgggttcttgcagtaggctcctcagcggtgtctaaacacgccactcaggtgattctatgcaccatcacattggaaacttttttcattgactgttacttaatgagaagacttccctccgggatggttctgaagcttccttcataggagcaagcctttggcggagagcactgagcagacgtgcagcatctttgctggcttctaccgaaacaccatggatccagacgtggttttgtggtcctgcacgtggaagccagccctgcgtggggtgagcctgggactgtgggcagagaacctcaagcaccgggccggcacccaagtgcagagactgcatcgtcccagcaggaggcgctgcttccaggctccctggacggactccgggagggcggcctttccccccagccccagaggtgggcctgcccttttccgagcatgggacacagcccaggaaaacgcatggcttgtcctccagacacaggtgctaacagggccgtcagacaagggccagggactcaggcttttaggaatttcagctccagagccaccatgcagtgggaccaggggtctgcgtggacaggaagcaagctgtgtagacgggggtccatgaagtagagacagggttttggggaaggttggggcagggcaaggaggaaaagccacatttacagcaatttctgaagtcttttcattttttccccctgaatcacgtccataataggatttgaatttaataaactgctgaaggttcctggccctgagtcccagtgtcctcccagcccccgcccagctgtgggtgtgcatggggagcggtacgagggagggtaaaatgggcccctgggacgccgcgtgcagagcagagatgaatggcccgaaaccctcgcgctgctctgcgcccttcgtcatccagtcggggtggttagggactgtcagagaaaaataatttagcggccatggctctaactgatgtgctgcattctggggtcaaatgacttttacaaagtagtagtgctgcctggtttctctatcgtgagagctcagggctgataacatgaaagaaaaaggcactgcagccagaattcactgacattcttcacatttcacatgagtgggacgcaggaggggggctggggagggtggagggatgtttcctgcttaacagattcaacagaagagtggcaggctcagctgggtgagcaaggtatcccagcgacgggggacacgccccagaccatgggtggtggggcttctcagaggaggtggcaggagacccgagcctgccaaggttgcacctaaggtcacgggcagcattaggagggctctctcccagtctccccacccccccgtcccccctcccccaggctgcaggggtgaagtggcttccaggacggtcactggcaagtttaagctacagagagtgtagaaacagggtgaaaaaggaagagagaggggagtaaataagaaggaggtgtaagaaaagaccaagccaggccccagcgcccttgtgaggaagtgcccagggacttatgtggaagccgtccttgctgtctgccaccttgtttttacttacattgtgtttttatttgagggcgagtttggacggcaagactgatggagattgtggtctaaatgcctctaacccactccttaaaatgaccaccggatgttccacaagtacttgaaaatgaatgaatggcttcccgagaggcagaaggcaggggtgtgccctaccccacgccggccaagagttcaacaagcattggttgacaagtgaatagtgagcacttgaacccagtcacaattcaagatgagggctctgccatgacgcatgtggtctgtgtcaccctgcagtctccctgagcagtgtctgaggttcgagtgggaccctacattcgtgaagagatttatcatctccccaggaaaaataacagattctgtcctaggtgttgtgatgtaacaatggtagcgatcacagccataacttacaattattgcatacttacgacgagtcccgcactgggctaagtgctttttaactatgtgaaatgtttctttccttgattgatgccaaaatgaataaagataattttctgtatctgct

SEQ ID NO: 2 - protein, 130 aa, Homo sapiens
MDPDVVLWSCTWKPALRGVSLGLWAENLKHRAGTQVQRLHRPSRRRCFQAPWTDSGRAAFPPSPRGGPALFRAWDTAQENAWLVLQTQVLTGPSDKGQGLRLLGISAPEPPCSGTRGLRGQEASCVDGGP

SEQ ID NO: 3 - DNA, 285 bp, Homo sapiens
  CDS (1)..(285)
atggatccagacgtggttttgtggtcctgcacgtggaagccagccctgcgtggggtgagcctgggactgtgggcagagaacctcaagcaccgggccggcacccaagtgcagagactgcatcgtcccaacaggaggcgctgcttccaggctccctggacggactccgggagggcggcctttccccccagccccagaggtgggcctgcccttttccgagcgtgggacacagcccaggaaaacgcatggcttgtcctccagacacagggcgagtttggacggcaagac

SEQ ID NO: 4 - protein, 95 aa, Homo sapiens
MDPDVVLWSCTWKPALRGVSLGLWAENLKHRAGTQVQRLHRPNRRRCFQAPWTDSGRAAFPPSPRGGPALFRAWDTAQENAWLVLQTQGEFGRQD

SEQ ID NO: 5 - DNA, 19 bp, Artificial Sequence
  Description of Artificial Sequenceprimer GSP1
ttatctttattcattttgg

SEQ ID NO: 6 - DNA, 23 bp, Artificial Sequence
  Description of Artificial Sequenceprimer GSP2
tgcgggactcgtcgtaagtatgc

SEQ ID NO: 7 - DNA, 20 bp, Artificial Sequence
  Description of Artificial Sequenceprimer AUAP
ggccacgcgtcgactagtac

SEQ ID NO: 8 - DNA, 19 bp, Artificial Sequence
  Description of Artificial Sequenceprimer RTR-5
ccattcattcattttcaag

SEQ ID NO: 9 - DNA, 17 bp, Artificial Sequence
  Description of Artificial Sequenceprimer RTF-6
aaaacgcatggcttgtc

SEQ ID NO: 10 - DNA, 25 bp, Artificial Sequence
  Description of Artificial Sequence_-actin reverse primer
agggtacatggtggtgccgccagac

SEQ ID NO: 11 - DNA, 25 bp, Artificial Sequence
  Description of Artificial Sequence_-actin forward primer
ccaaggccaaccgcgagaagatgac

SEQ ID NO: 12 - DNA, 127 bp, Homo sapiens
  fragment of sequence AQ548392,  nuclotide 1 correspond to nucleotide 304 and nucleotide 127 correspond to nucleotide 430 of the complete sequence
tggacccccgtctacacagcttgcttcctgtccactcagacccctggtcccactgcatggtggctctggagctgaaattcctaaaagcctgagtccctggcccttgtctgacggccctgttagcacc